(19) United States
(12) Patent Application Publication
(10) Pub. No.: US 2026/0096835 A1
(43) Pub. Date: Apr. 9, 2026

(54) Anti-torque Features for External Fixation Frame Components

(71) Applicant: Stryker European Operations Limited, CARRIGTWOHILL (IE)

(72) Inventor: Philippe Lehmann, Lamboing (CH)

(73) Assignee: Stryker European Operations Limited, CARRIGTWOHILL (IE)

(21) Appl. No.: 19/338,043

(22) Filed: Sep. 24, 2025

Related U.S. Application Data
(60) Provisional application No. 63/703,283, filed on Oct. 4, 2024.

(51) Int. Cl. A61B 17/62; A61B 17/00

(52) U.S. Cl. CPC A61B 17/62; A61B 2017/00477

(57) ABSTRACT

An external fixation system may include two fixation rings, struts, a threaded bolt, and a post having a base, a free end, and first and second side walls. A hole may extend through the post, and a threaded hole may have an opening positioned in the base and extending toward the free end of the post. A first protrusion may protrude from the base in a direction opposite the free end of the post. When the system is assembled, the struts couple the first and second rings, the base of the post contacts a top surface of the first ring, and the bolt extends through one of the first plurality of holes from a bottom surface of the first ring into the threaded hole of the post to secure the post to the first ring such that the first protrusion penetrates the top surface of the first ring.

### CROSS-REFERENCE TO RELATED APPLICATIONS

This application claims priority to the filing date of U.S. Provisional Patent Application No. 63/703,283, filed October 4, 2024, the disclosure of which is hereby incorporated by reference herein.

### BACKGROUND OF THE DISCLOSURE

The present disclosure relates to systems and components of external fixation frames. More particularly, the present disclosure relates to accessory devices having anti-torque features that can be coupled to rings of an external fixation frame.

Many different types of bone deformities can be corrected using external fixation systems to perform the distraction osteogenesis process. For example, an Ilizarov device or similar external fixation system may be used. Such systems generally use rings also designated as fixation plates connected by threaded rods or struts for manipulation, lengthening, angulation, rotation, and/or translation of deformities of bones.

As the struts are manipulated, the rings or fixation plates change positions relative to one another, causing the bones or bone segments attached to the fixation plates to change positions relative to one another, until the bone segments are in a desired position relative to one another. Such fixation systems typically include accessory devices, such as pins or other fixation members, that mechanically couple the rings to the bone. Fixation systems have many areas which may be improved including, for example, the way in which accessory devices are secured to the ring(s) of the fixation system.

### BRIEF SUMMARY

According to one aspect of the disclosure, An external fixation system may include a first fixation ring having a first plurality of holes extending therethrough, a second fixation ring having a second plurality of holes extending therethrough, and a plurality of struts configured to couple the first fixation ring to the second fixation ring. The system may include a post having a base, a free end opposite the base, first and second side walls each extending from the base to the free end, at least one hole extending from the first wall to the second wall through the post, a threaded hole having an opening positioned in the base and extending toward the free end of the post, and a first protrusion protruding from the base in a direction opposite the free end of the post. The system may also include a threaded bolt. In an assembled condition of the external fixation system, the plurality of struts couple the first fixation ring to the second fixation ring, the base of the post is in contact with a top surface of the first fixation ring, and the threaded bolt extends through one of the first plurality of holes from a bottom surface of the first fixation ring into the threaded hole of the post to secure the post to the first fixation ring such that the first protrusion penetrates the top surface of the first fixation ring. A second protrusion may protrude from the base in the direction opposite the free end of the post, and the first and second protrusions may be positioned on opposite side of the opening of the threaded hole. In the assembled condition of the external fixation system, the second protrusion penetrates the top surface of the first fixation ring. The first protrusion and the second protrusion may each extend along a first imaginary line that also passes through a center of the opening positioned in the base. A third protrusion and a fourth protrusion may protrude from the base in the direction opposite the free end of the post, the third and fourth protrusions being positioned on opposite sides of the opening of the threaded hole. The third protrusion and the fourth protrusion may each extend along a second imaginary line that also passes through the center of the opening positioned in the base, the first imaginary line being perpendicular to the second imaginary line. The base may include a first flat edge and a second flat edge parallel to the first flat edge, a first curved edge connecting the first flat edge and the second flat edge, and a second curved edge connecting the first flat edge and the second flat edge. The first, second, third, and fourth protrusions may each terminate in a first end that is positioned along the first or second curved edge at a location adjacent to the first or second flat edge. The first, second, third, and fourth protrusions may each terminate in a second end that is a located a spaced distance from the opening positioned in the base. The first fixation ring and the second fixation ring may each be formed of carbon fiber. The first fixation ring and the second fixation ring may each be formed of aluminum. The post may be a pin post, and the system may include at least one pin configured to be received in the pin post and extend into bone of a patient. The system may also include a connector, the connector being configured to be received within the at least one hole of the post. The system may also include at least one pin configured to be received in the connector and extend into bone of a patient. The system may also include at least one wire configured to be received in the connector and extend through bone of a patient. In the assembled condition of the external fixation system, the first and second protrusions may penetrate the top surface of the first fixation ring such that a bottom surface of the base is substantially flush with the top surface of the first fixation ring. During assembly of the external fixation system, the threaded bolt may be configured to be tightened between about 11 Nm and about 17 Nm into the threaded hole of the post without deforming the one of the first plurality of holes.

According to another aspect of the disclosure, a method of assembling an external fixation system may include coupling a first fixation ring to a second fixation ring via a plurality of struts, the first fixation ring having a first plurality of holes extending therethrough and the second fixation ring having a second plurality of holes extending therethrough. The method may include positioning a base of a post on a top surface of the first fixation ring so that an opening in the base of the post aligns with one of the first plurality of holes. The method may include passing a threaded bolt through the one of the first plurality of holes from a bottom surface of the first fixation ring toward the top surface of the first fixation ring until the threaded bolt is at least partially received within the opening in the base of the post. The method may also include rotating the threaded bolt to engage threads of the threaded bolt with complementary threads of a threaded hole of the post extending from the opening in the base of the post in a direction toward a free end of the wire post to secure the post to the first fixation ring. During the rotating, a first protrusion extending from the base in a direction opposite the free end may contact the top surface of the first fixation ring, and the rotating may be continued until the first protrusion penetrates the top surface of the first fixation ring. During the rotating, the first protrusion and a second protrusion extending from the base in the direction opposite the free end may contact the top surface of the first fixation ring at locations on opposite sides of the one of the first plurality of holes, and the rotating may be continued until both the first and second protrusions penetrate the top surface of the first fixation ring. The first protrusion and the second protrusion may each extend along a first imaginary line that also passes through a center of the opening in the base. The post may include a third protrusion and a fourth protrusion protruding from the base in the direction opposite the free end of the post, the third and fourth protrusions being positioned on opposite sides of the opening in the base. The third protrusion and the fourth protrusion may each extend along a second imaginary line that also passes through the center of the opening in the base, the first imaginary line being perpendicular to the second imaginary line. The base may include a first flat edge and a second flat edge parallel to the first flat edge, a first curved edge connecting the first flat edge and the second flat edge, and a second curved edge connecting the first flat edge and the second flat edge. The first, second, third, and fourth protrusions may each terminate in a first end that is positioned along the first or second curved edge at a location adjacent to the first or second flat edge. The first, second, third, and fourth protrusions may each terminate in a second end that is a located a spaced distance from the opening in the base. The first fixation ring and the second fixation ring may each be formed of carbon fiber. The first fixation ring and the second fixation ring may each be formed of aluminum. The post may be a pin post, and the method may include inserting at least one pin through the pin post and into bone of a patient. The metho may include coupling a connector to the post via a hole in the post. The method may include inserting at least one pin through the connector and into bone of a patient. The method may include inserting at least one wire through the connector and through bone of a patient. After rotating the threaded bolt, the first and second protrusions may penetrate the top surface of the first fixation ring such that a bottom surface of the base is substantially flush with the top surface of the first fixation ring. The step of rotating the threaded bolt may include tightening the threaded bolt between about 11 Nm and about 17 Nm without deforming the one of the first plurality of holes.

### BRIEF DESCRIPTION OF THE DRAWINGS

FIG. 1 is a perspective view of an external fixation system of the prior art.

FIGS. 2A-C are different views of a female post according to the prior art.

FIG. 2D is a view of a connecting bolt according to the prior art.

FIG. 2E is an exploded view of the female post of FIGS. 2A-C being coupled to a ring of the external fixation system of FIG. 1 using the connecting bolt of FIG. 2D, with a connector being coupled to the female post using a nut.

FIG. 2F is a perspective view showing the connector of FIG. 2E having been coupled to the ring via the female post 210, and a half-pin having been secured within the connector.

FIG. 2G shows an alternate version of FIG. 2F in which a wire, instead of a half-pin, has been secured within the connector.

FIGS. 3A-D are different views of a female post according to an aspect of the disclosure.

FIGS. 4A-D are different views of a female post according to another aspect of the disclosure.

FIGS. 4E-F show a five-opening version of the two-opening female post of FIGS. 4A-D.

FIG. 4G shows a bottom surface of an alternate version of the female post of FIGS. 4E-F.

FIGS. 5A-D are different views of a pin post according to an aspect of the disclosure.

FIG. 6A is a side view of a portion of a strut.

FIG. 6B is an enlarged perspective view of a joint of the strut of FIG. 6A.

FIG. 7A is a perspective view of a twisted plate.

FIG. 7B is a perspective view of the plate of FIG. 7A coupled to a ring and to a threaded rod and connector accessory.

FIG. 8 is a perspective view of an angled pin connector.

FIGS. 9A-9B are perspective views of an example of an angled pin adaptor.

FIG. 10A is a perspective view of a connection plate.

FIG. 10B is a perspective view of connection plates shown in FIG. 10A coupled to a fixator system.

### DETAILED DESCRIPTION

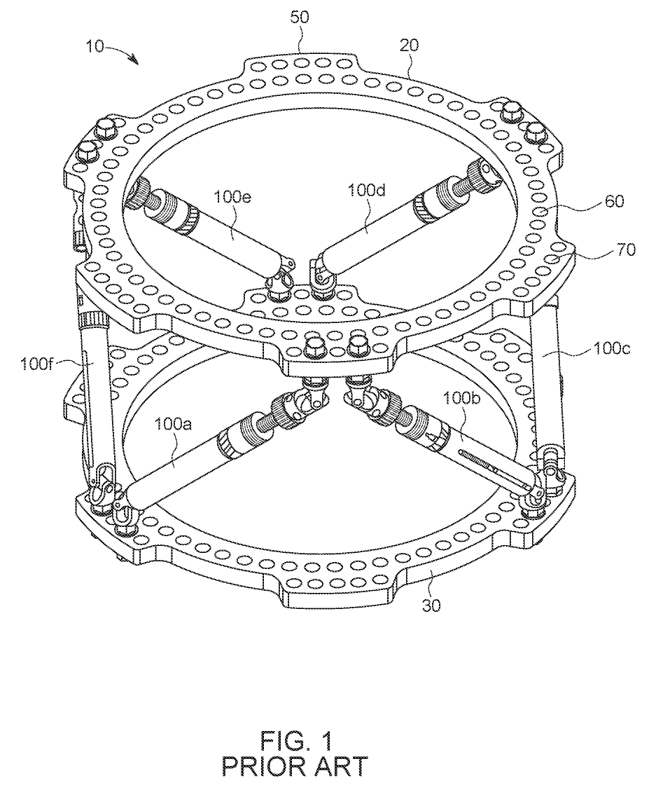

FIG. 1 shows an external fixation frame 10 in an assembled condition according to one aspect of the disclosure. Generally, fixation frame 10 includes a first ring 20 and a second ring 30, with six adjustable length telescopic struts 100a-f coupling the first ring 20 to the second ring 30. The first ring 20 may also be referred to as a proximal ring or a reference ring, while the second ring 30 may also be referred to as a distal ring or a moving ring. In the illustrated embodiment, each strut 100a-f includes a threaded portion that may thread into or out of a tube portion, for example by interaction with quick release mechanism 130, to decrease or increase the length, respectively, of the telescopic strut. Each end of each strut 100a-f may be coupled to the first ring 20 and second ring 30 via a joint mechanism, such as a ball joint, a constrained hinge joint, or a universal joint as illustrated. The use of universal joints on each end of the strut provides for six degrees of freedom of motion of the external fixation system 10. It should be understood that although the disclosure is generally described in the context of closed circular rings, the concepts described herein may apply with equal force to other types of rings, such as open rings and/or U-shaped rings.

In external fixation system 10, telescopic struts 100a-f are used to reduce fractures and correct deformities over time. Patients correct the deformities by prescribed adjustments of the struts 100a-f. The lengths of the struts 100a-f are adjusted over time to change the position and orientation of the two rings 20, 30 with respect to one another, which in turn repositions and reorients the bone fragments, with a goal of correcting the bone deformity. The adjustment of the external fixator 10 should strictly comply with the predetermined correction plan.

Rings 20 and 30 of external fixation system 10 may include a plurality of extension tabs 50. In the illustrated example, each ring 20 and 30 includes six extension tabs 50 spaced circumferentially around the perimeter of the respective rings, although more or fewer may be suitable depending on the particular components of the fixation system. In addition to what is described directly below, extension tabs 50 may help increase the cross-sectional area of rings 20, 30 and thus provide for increased stiffness of the rings.

With this configuration, each ring 20, 30 includes a first inner circumferential row of holes 60 and a second outer circumferential row of holes 70. As illustrated, the second outer circumferential row of holes 70 may be only positioned on the plurality of extension tabs 50 on the rings 20 and 30. It should be understood that although the second outer circumferential row of holes 70 is shown in FIG. 1 as being positioned solely on extension tabs 50, top ring 20 and/or bottom ring 30 may contain two complete rows of holes, for example with a completely circular (or nearly completely circular) geometry. The use of extension tabs 50, compared to two full circumferential rows of holes, may help reduce overall bulk of rings 20, 30 and also provide for intuitive strut placement for surgical personnel. The completely circular version of rings 20, 30 with two full (or nearly full) rows of circumferential holes may be particularly suited for relatively small diameter rings, although indentations or other features may be introduced to provide an intuitive interface for strut placement by surgical personnel. Further, in the illustrated embodiment, the first and second circumferential rows of holes 60 and 70 are positioned so that the first row of holes 60 does not align radially with the second row of holes 70. In other words, the first row of holes 60 has a staggered configuration with respect to the second row of holes 70. The additional hole options may also be utilized for connecting other components, such as fixation pins to couple the rings 20, 30 to the respective bone fragments. Still further, the staggered configuration of holes between the first and second rows 60, 70 may also help prevent interference between components attached to nearby holes, for example such as a strut 100a-f positioned in a first hole and a fixation pin or other fixation member attached to an adjacent or nearby second hole. For example, a relatively thin wire extending radially from one of the holes in the first circumferential row 60 may not radially interfere with a hole positioned in the second circumferential row 70 because of the radial staggering. It should be understood that the size of the tabs 50 may increase or decrease depending on the diameter of the rings 20 and 30, with greater diameter rings 20 and 30 having larger tabs 50 with more holes 70 compared to smaller diameter rings. For example, the illustrated tabs 50 include six holes 70, and a smaller ring may include smaller tabs with four holes each, for example.

In order to correct a bone deformity, a physician may position the patient’s bone through the rings 20, 30 and couple the first ring 20 to a first bone segment and the second ring 30 to a second bone segment, for example using bone fixation pins. Over time, the physician, patient, or other individual may adjust the lengths of the various struts 100a-f, for example by following a correction schedule, to cause the rings 20, 30 to shift positions (e.g. distance, angle) relative to each other. Because the rings 20, 30 are each coupled to their respective bone segments, movement of the rings 20, 30 relative to each other results in movement of the bone segments relative to each other, eventually correcting the bone deformity.

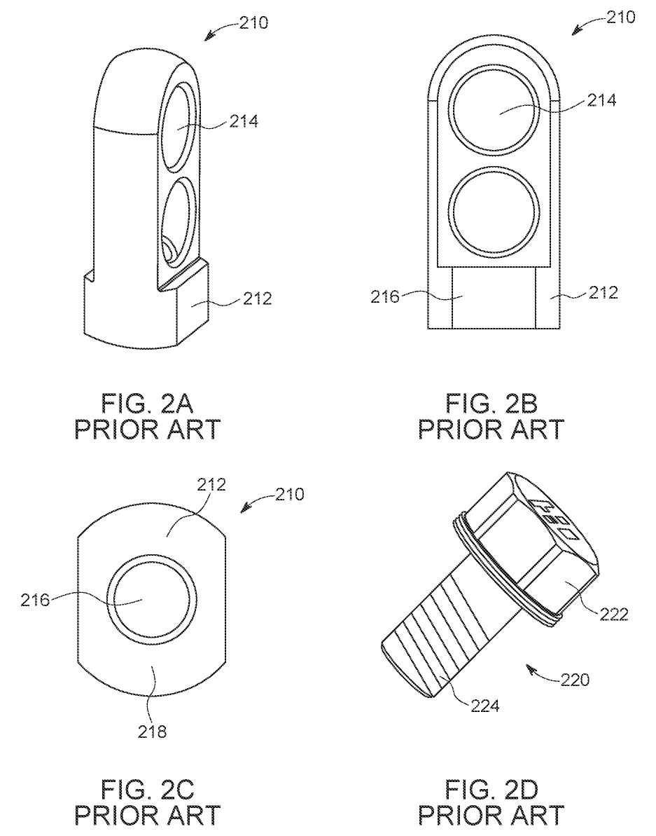

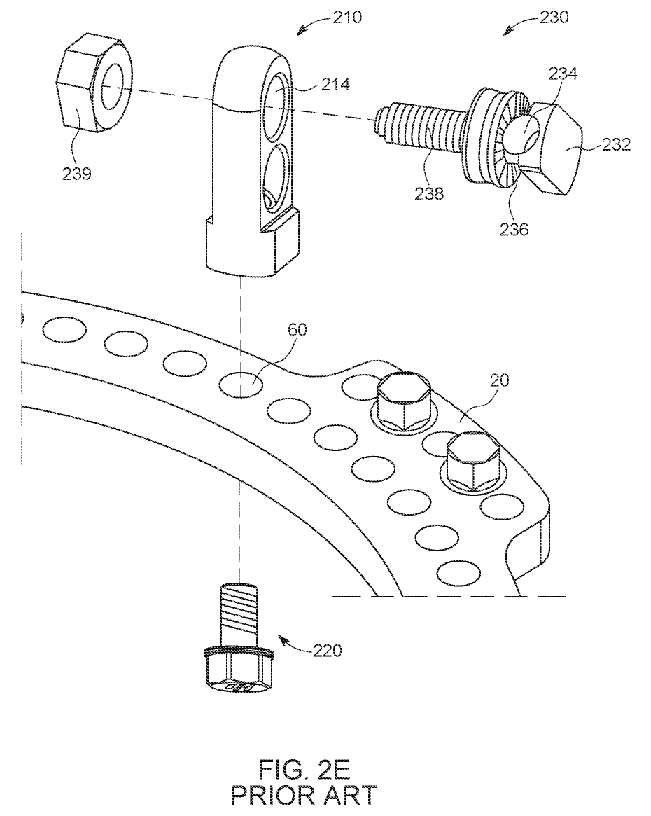

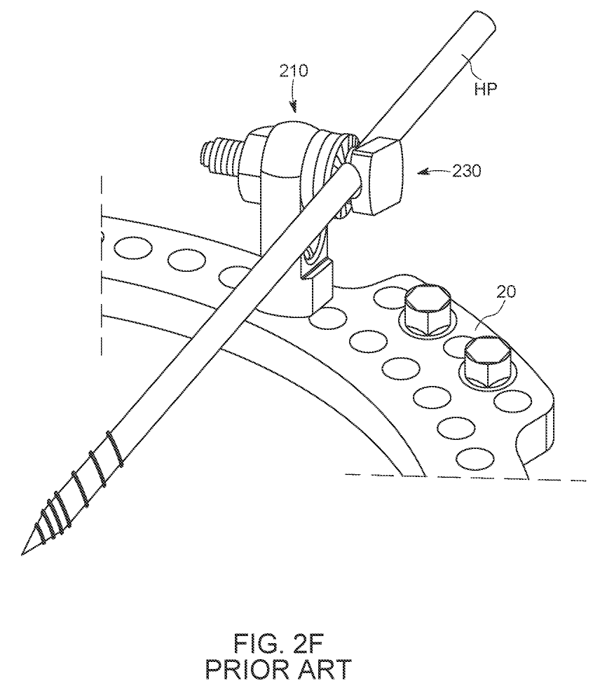

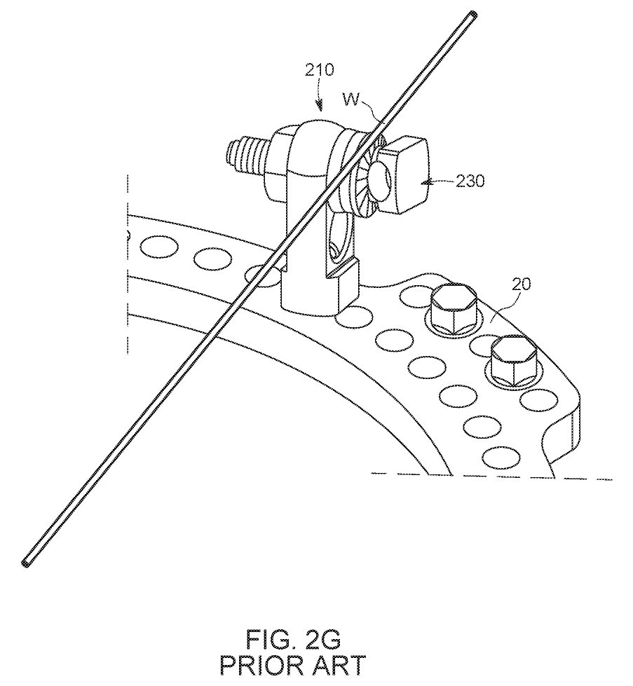

FIGS. 2A-C are perspective, cross-sectional, and bottom views, respectively, of a female post 210. Female post 210 may be configured to couple to one of the rings 20, 30 via one of the holes 60, 70, with the female post 210 configured to receive one or more accessory components to help fix the respective ring 20, 30 to the desired bone fragment. For example, female post 210 may include a base 212 with a base surface 218 configured to be in contact with a top or bottom surface of one of the rings 20, 30. The base 212 may include an opening 216 configured to align with a respective opening 60, 70 in one of the rings 20, 30. The female post 210 may include a main post member extending upwardly from the base 212 (opposite the base surface 218), and the main post member may include one or more openings 214 formed therethrough. Although two openings 214 are shown, the female post 210 may include as few as one opening 214, or three, four, five, or more openings 214. The openings 214 are preferably vertically aligned with each other. FIG. 2D shows an example bolt 220 that may be used to couple the female post 210 to a ring 20, 30. Bolt 220 may include a head 222 (e.g. a hex shape head) and a shaft with threading 224. Although not shown in FIG. 2B, the opening 216 in the female post 210 may be defined by an internal surface that includes threading that is complementary to that of the threading 224 of the bolt 220. In some examples, female post 210 (and other related accessories described herein such as a pin post) may be formed of stainless steel or another similar material. The female post 210 (or other related accessory described herein such as a pin post) may be formed of other suitable materials, particularly materials that are harder than the material forming the rings 20, 30.

Referring now to FIG. 2E, in one example, the female post 210 may be positioned on the top of ring 20 with bolt 220 passing through hole 60 from the bottom of the ring 20, with the bolt 220 being rotated to thread into opening 216 to tightly connect the female post 210 to the ring 20. One of the openings 214 of the female post 210 is shown as being utilized in FIG. 2E to couple a connector 230 to the ring 20 via the female post 210. In the particular example shown, the top opening 214 of female post 210 is being utilized, but any opening may be utilized (including for embodiments in which the female post has one, three, four, five or more openings), to receive connector 230. In this particular embodiment, connector 230 is a component that includes a head 232 that defines an opening 234 that is sized and shaped to receive a half-pin HP therethrough. However, a shoulder 236 may be defined between the head 232 and a collar of the connector 230, with the shoulder 236 being sized and shaped to receive a wire W therethrough. The connector 230 may include a threaded shaft 238 sized to pass through opening 214, and a nut 239 may be used to secure the connector 230 to the female post 210. In some examples, upon tightening the nut 239, the wire W received within shoulder 236 and/or the half-pin HP received within the hole 234, becomes clamped tightly to the connector 230.

FIG. 2F shows an example in which the connector 230 has been coupled to the ring 20 via the female post 210, and a half-pin HP has been secured within the connector 230. The half-pin HP may have a threaded distal end, which may be sharp or blunt and may have features for self-drilling. Although not shown in FIG. 2F, the tip of the half-pin HP may be inserted into the bone fragment that is being attached to the ring 20. Although a single half-pin HP is shown in FIG. 2F, it should be understood that more than one half-pin may be used, including one or more on each ring 20, 30, to help firmly connected the respective bone fragment to the respective ring 20, 30.

FIG. 2G shows an example in which the connector 230 has been coupled to the ring 20 via the female post 210, and a wire W has been secured within the connector 230. The wire W may be smooth. Although not shown in FIG. 2G, the wire W be inserted through the bone fragment (or through other tissue) that is being attached to the ring 20, with the opposite end of the wire W passing through the tissue and into a corresponding connector (not shown) on the opposite end of the ring 20 that receives the wire W, such that the wire W is secured on each end of the ring 20. The wire W may be tensioned, for example via a wire tensioner, which is known in the art.

Regardless of the specific accessory being used to connect to the female post 210, certain concerns may arise relating to torque and the securement of the female post 210 to the ring 20 via bolt 220. For example, when threading bolt 220 into female post 210, an anti-torque tool (e.g., an anti-torque wrench) may need to be used to secure the rotational position of the female post 210 relative to the ring 20 while the bolt 220 is being rotated, otherwise rotation of the bolt 220 may result in the female post 210 undesirably rotating, instead of resulting in the bolt 220 threading into the female post 210. This additional step requires more time and may require more tools (e.g. an anti-torque tool), resulting in overall greater cost and complexity. Further, when connecting the connector 230 (or any other accessory) to the female post 210, significant forces may be needed, such as when threading the nut 239 onto the threaded shaft 238. Similarly, when driving the half-pin HP into the tissue and/or when tensioning the wire W, forces may be applied on the female post 210 via the connector 230. Such forces, depending on the directionality of the forces, could tend to rotate the female post 210 in a direction so that the bolt 220 may loosen, and even a small loosening of the bolt 220 may negatively affect the stability of the female post 210 and this all components coupled to the female post 210. Still other forces may tend to be applied to the female post 210, for example based on loads applied from patient movement or weight of the patient bearing on the half-pin HP or wire W. These forces may similarly translate to the female post 210 with the potential for loosening the connection between the female post 210 and bolt 220, which may tend to destabilize the construct.

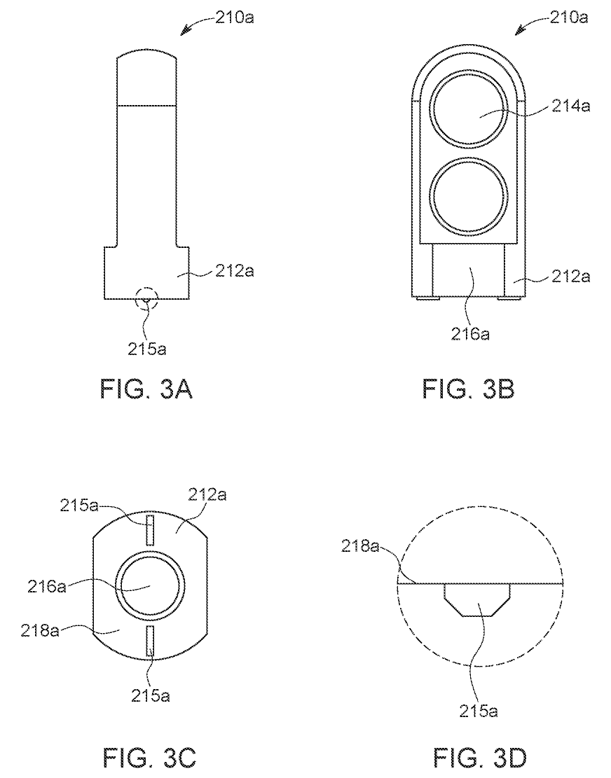

FIGS. 3A-D are different views of a female post 210a (or portions thereof) that is identical to female post 210 with a single exception, which is the inclusion of friction-enhancing elements, which may be referred to as protrusions or teeth 215a, configured to enhance the rotational stability of the female post 210a relative to the ring 20. As with female post 210, female post 210a may include a base 212a defining a bottom surface 218a and an opening 216a to receive a bolt 220, and a main post portion including one or more openings 214a to receive accessories therethrough. The description of female post 210 provided above, including alternative options, fully apply to female post 210a and are thus not described again here.

As noted above, the only difference in female post 210a is the inclusion of teeth 215a protruding from the bottom surface 218a of the base 212a. In the specific example shown in FIGS. 3A-C, two teeth 215a are provided, with each tooth 215a extending in a direction orthogonal to the central longitudinal axes of the holes 214a. Stated otherwise, the two teeth 215 extend in a direction that is generally parallel to the two flat edges of the base 212a and the main post body. Further, each tooth 215a extends in a direction that, if continued, would pass through the radial center of hole 216a and overlap the other tooth 215a.

FIG. 3D is an enlarged view of one of the teeth 215a, with the dashed circle of FIG. 3D being an enlarged version of the dashed circle of FIG. 3A. In this example, each tooth 215a has two lateral edges extending away from the bottom surface 218a, with these two lateral edges each transition to a diagonal edge (about 45 degrees) to a flat bottom surface. This construction provides for a relatively sharp tip that is capable of digging into the surface of the ring 20, 30 to enhance frictional engagement (and provide enhanced resistance against rotation). The teeth 215a do not hinder the bottom surface 218a of the female post 210a from fully contacting the relevant surface of the ring 20, 30. For example, when bolt 220 is threaded into the opening 216a of female post 210a through a hole 60 of ring 20 (for example a carbon fiber version of ring 20), and tightened to a prescribed torque of between about 8 Nm and about 14 Nm (including about 9 Nm, about 10 Nm, about 11 Nm, about 12 Nm, or about 13Nm), the teeth 215a become fully “stamped” into the ring 20 so that the remainder of the surface 218a can fully confront the relevant surface of the ring 20. In other words, the inclusion of teeth 215a does not result in any gap between the surface of the ring 20 and the bottom surface 218a of the base 212a of the female post 210a when tightened to a prescribed amount. Even with overtightening, which was tested at a 50% tightening above the prescribed amount, there was no deformation of the holes 60 of the ring 20 or other observed negative effects.

An embodiment of a female post identical to that shown in FIGS. 3A-3D, but with five vertically aligned openings instead of two vertically aligned openings 214a, was tested to determine performance in terms of resistance to counter-rotation. In particular, six separate female posts 210a (with five openings 214a each) were coupled to a carbon fiber version of ring 20, with the bolt 220 securing the female post 210a to the ring 20 by being tightened to a threshold torque. A threaded shaft was inserted through the second opening 214a (the first opening 214a being the one closest to the base 212a, the fifth opening 214a being the one farthest from the base 212a), and the shaft was secured to the female post 210a using nuts on the threaded shaft on each side of the female post 210a. The ring 20 was secured to a fixture and an increasing load was applied on the threaded shaft a selected distance from the center of the female post 210a, such that the threaded shaft acted as a lever arm having a selected distance. Force was applied in the “unscrewing” direction that would tend to loosen the female post 210a from the bolt 220. Force was applied until the female post 210a began to “slip” (e.g. rotate) relative to the ring 20. For the six tests performed, the mean force or mean moment at which the female post 210a began to slip was determined.

One relevant example of moments is described below. When a wire W is used, as noted above it is tensioned by a wire tensioner. In some examples, the nominal wire tension given by the wire tensioner may be represented by W_{T}. A typical or example patient load may be represented by P_{L}. Measurements were performed on a 3-wire construct which revealed that approximately 40% of the load applied by the patient P_{L} may be transmitted to the wire W with the highest tension. A final tension force T_{F} on the wire W may be estimated as W_{T} plus 40% of the patient load P_{L}, resulting in a total estimated tension force T_{F} on the wire W. In the tested example, given the relevant lever arm, a rotation moment R_{M} applied on the wire post (e.g. the connector 230) was determined. Notably, this rotation moment R_{M} was larger than the mean moment from testing described above at which the female post 210a begin to slip, suggesting that, at least in some cases, even with the additional security provided by the two teeth 215a, the specific embodiment of female post 210a shown in FIGS. 3A-3D may be at risk of loosening from the bolt 220. As is described in greater detail below, the addition of more teeth and/or differently oriented teeth may provide enhanced securement.

Before describing the further embodiment with additional teeth, further tests were performed to compare the resistance to rotation of female posts 210 compared to female posts 210a. For example, the same test as described immediately above was performed, for five-opening versions of female post 210 (a total of six tests were performed for this tooth-less embodiment) as well as for five-opening versions of female post 210a (a total of six tests were performed for this toothed embodiment), with the relevant ring 20 being formed as an aluminum ring instead of a carbon fiber ring. For the female post 210a with teeth 215a, the six tests were determined to have a mean force at the point of slippage, with a mean moment calculated using the relevant lever arm. For the female post 210 without teeth, the six tests had a calculated mean force at the point of slippage, with a mean moment calculated based on the relevant lever arm. Notably, the version of female post 210a with teeth 215a showed between about 15% to about 20% better resistance (which was determined to be a statistically significant enhancement) to slippage from torque compared to the version of female post 210 without the teeth, demonstrating the clear enhancement in stability provided by the teeth 215a. However, as with the carbon fiber ring 20, the mean moment for the female post 210a with teeth 215a at which slippage occurred is lower than the example rotation moment R_{M} threshold described above, meaning that slippage may be a concern.

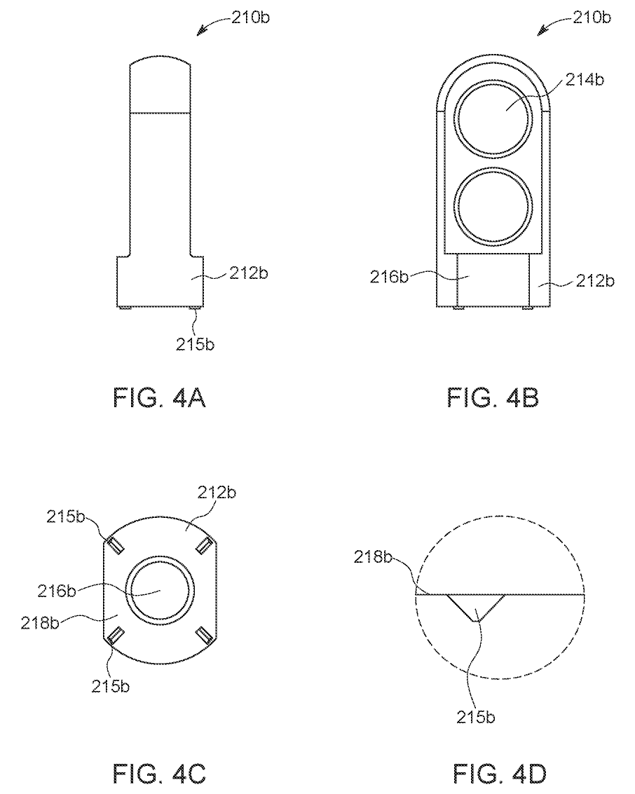

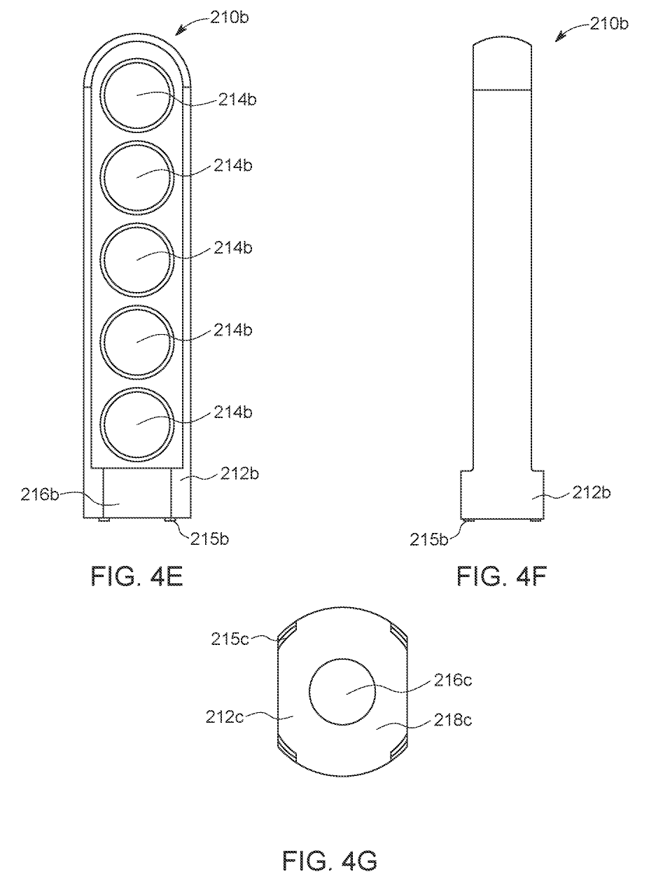

FIGS. 4A-D are different views of a female post 210b (or portions thereof) that is a version of female post 210a but with additional friction-enhancing elements (e.g. protrusions or teeth 215b). As with female posts 210, 210a, female post 210b may include a base 212b defining a bottom surface 218b and an opening 216b to receive a bolt 220, and a main post portion including one or more openings 214b to receive accessories therethrough. The descriptions of female posts 210, 210a provided above, including alternative options, fully apply to female post 210b (other than the configuration of teeth 215b) and are thus not described again here.

As noted above, the only difference in female post 210b is the number and configuration of teeth 215b protruding from the bottom surface 218b of the base 212b. In the specific example shown in FIGS. 4A-C, four teeth 215b are provided, with each tooth 215b extending in a direction that, if continued, would pass through the center of opening 216b. The four teeth 215b may be provided at about 90 degree intervals, with a first line passing through one pair of the teeth 215b, and a second line passing through the other pair of the teeth 215b, with the first and second lines being perpendicular to each other. In the illustrated embodiment, each tooth 215b has a first end that is positioned or adjacent to a location where one of the two rounded surfaces of the base 212b transitions into one of the two flat surfaces of the base 212b, and a second end that is a space distance from the opening 216b.

FIG. 4D is an enlarged view of one of the teeth 215b. The view of FIG. 4B is an end view, as if looking at the point where one of the flat surfaces of the base 212b meets an adjacent rounded surface of the base 212b, with the bottom surface 218b of the base 212b oriented upward and at eye level. In this example, each tooth 215b has two side edges extending away from the bottom surface 218b toward each other at an angle of about 45 degrees, with a short flat edge joining the ends of the two side edges to form a point (or a slightly flattened point). This construction provides for a relatively sharp tip that is capable of digging into the surface of the ring 20, 30 to enhance frictional engagement (and provide enhanced resistance against rotation). As with teeth 215a, the teeth 215b do not hinder the bottom surface 218b of the female post 210b from fully contacting the relevant surface of the ring 20, 30. For example, when bolt 220 is threaded into the opening 216b of female post 210b through a hole 60 of ring 20 (for example an aluminum version of ring 20), and tightened to a prescribed torque of between about 8 Nm and about 14 Nm (including about 9 Nm, about 10 Nm, about 11 Nm, about 12 Nm, or about 13 Nm), the teeth 215b become fully “stamped” into the ring 20 so that the remainder of the surface 218b can fully confront the relevant surface of the ring 20. In other words, the inclusion of teeth 215b does not result in any gap between the surface of the ring 20 and the bottom surface 218b of the base 212b of the female post 210b when tightened to the prescribed amount. Even with overtightening, which was tested at a 50% tightening above the prescribed amount, there was no deformation of the holes 60 of the ring 20 or other observed negative effects. Although tooth 215a is shown in FIG. 3D with a shape that is slightly different than the shape of tooth 215b in FIG. 4D, it should be understood that either shape may be used for any of the teeth described herein.

A five-opening version of female post 210b (e.g. with five openings 214b aligned vertically instead of two openings 214b as shown in FIGS. 4A-C) was subjected to the same testing as described above for female post 210a. A five-opening version of female post 210b is illustrated in FIGS. 4E-4F for reference, although as noted above other numbers of holes may be provided using the same overall design as shown in FIGS. 4A-F (including the same number and configuration of teeth 215b). Referring to the testing that was performed, six separate female posts 210b (with five openings as shown in FIGS. 4E-F) were coupled to an aluminum version of ring 20, with the bolt 220 securing the female post 210b to the ring 20 by being tightened to a prescribed threshold torque. A threaded shaft was inserted through the second opening 214b (the first opening 214b being the one closest to the base 212b, the fifth opening 214b being the one farthest from the base 212b), and the shaft was secured to the female post 210b using two nuts on the threaded shaft on each side of the female post 210b. An increasing load was applied on the threaded shaft a selected distance from the center of the female post 210b, such that the threaded shaft acted as a lever arm having a selected distance. Force was applied in the “unscrewing” direction that would tend to loosen the female post 210b from the bolt 220. Force was applied until the female post 210b began to “slip” (e.g. rotate) relative to the ring 20. For the six tests performed, the median force or mean moment at which the female post 210b began to slip was determined.

Compared to the same testing performed on female post 210a on an aluminum version of ring 20, female post 210b provided between about 90% and about 100% more resistance to rotation than female post 210a with two teeth 215b, and between about 120% and about 130% more resistance to rotation than the female post 210 without teeth. Notably, the mean moment required to cause slippage of the female post 210b is significantly larger than the example rotation moment R_{M} threshold described above, meaning that slippage may no longer be a meaningful concern when using female posts 210b.

The same test described above for female posts 210b with an aluminum version of the ring 20 were also performed with a carbon fiber version of the ring 20. In that test, the median force (among the six test samples) resulting in slippage of the female post 210b relative to the carbon fiber ring 20 was determined, and a median moment was determined (based on a selected lever arm) that would result in the slippage. Once again, the mean moment required to cause slippage of the female post 210b with the carbon fiber ring 20 was determined to be larger than the example rotation moment R_{M} threshold described above, meaning that slippage may no longer be a meaningful concern when using female posts 210b, whether on an aluminum or carbon fiber ring 20.

FIG. 4G illustrates the bottom surface an alternate version of female post 210b, which may be identical to female post 210b with the exception of the configuration of the four teeth 215c protruding from the bottom surface 218c of the base 212c. Rather than extending in a direction that is radially outward form the center of the opening like teeth 215b, teeth 215c extend generally tangentially. In other words, the teeth 215c may follow a curvature of the curved sides of the bottom surface 218c and extend to the flat side surface of the base 212c. Female posts with these tangential teeth 215c were tested in the same manner as the female posts 210b with the radial teeth 215b, but provided less stability than the radial teeth 215b.

As should be understood from the above description, the inclusion of four teeth 215b may provide greater resistance to rotation and/or stability compared to the inclusion of two teeth 215a, and the inclusion of either two teeth 215a or four teeth 215b may provide greater resistance to rotation and/or stability compared to the inclusion of no teeth. One reason that four teeth 215b may provide enhanced stability compared to two teeth 215a is the simple fact that there are more teeth to provide contact and/or friction with the ring 20. However, the positioning of the teeth may also matter, as evidenced by four radial teeth 215b providing enhanced stability compared to four tangential teeth 215c. And although embodiments of two and four teeth are shown, it should be understood that other numbers and positions of teeth may be used, including as few as a single tooth, as many as three teeth, or as many as five or more teeth.

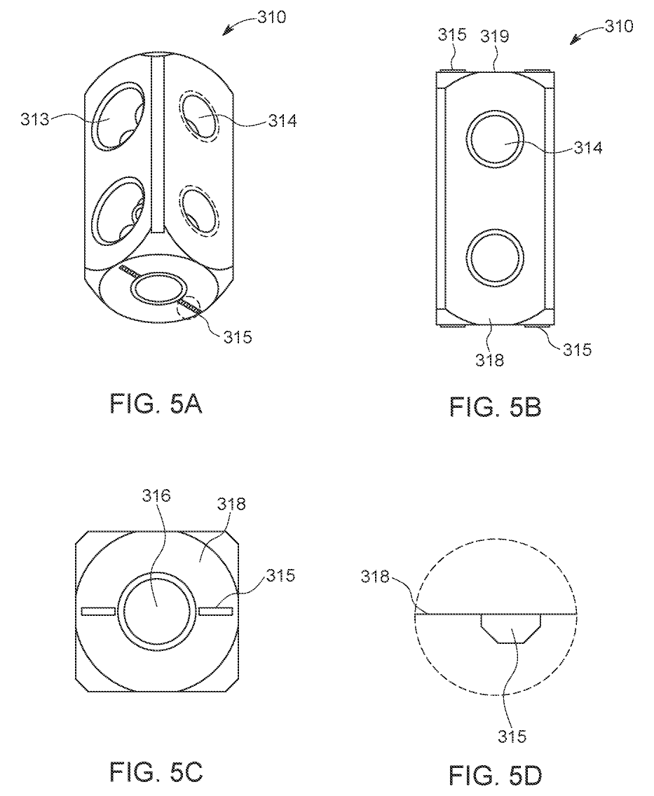

Although the embodiments described above focus on friction-enhancing structures (e.g. teeth) for use with a female post 210, it should be understood that these friction-enhancing structures may be applied to any other accessory device that is to be coupled to a ring 20, 30 of an external fixation frame. For example, although posts 210 are shown as female posts, there are also male versions of these posts which include a threaded shaft that passes through the hole 60 of the ring 20, with a nut or similar item securing the male post via threading over the threaded shaft. Those male posts may include any of the teeth described above for the female post to provide similar or identical benefits of enhanced stability and the ability to secure the posts to the ring 20 without needing to use an anti-torque tool such as an anti-torque wrench. Other types of posts may also benefit from these friction-enhancing structures. For example, FIGS. 5A-5C illustrate another accessory for use with an external fixation frame, which may be referred to as a pin post (although they have also been referred to as “rancho cubes”). Pin post 310 may include large through-holes 313 (two shown in the embodiment of FIGS. 5A-C, but pin posts may be provided with one or more than two holes, including three, four, five or more holes) which are configured to receive a pin, such as half-pin HP, therethrough. In some examples, the large through-holes are configured to receive centering sleeves first, with the pins being inserted through the centering sleeves received within the through-holes 313. The large through-holes 313 may extend through opposing faces of the pin post 310. Pin post 310 may also include additional through-holes 314, which are typically smaller through-holes, which can be threaded and configured to receive a set screw or similar compression member therethrough which may be configured to help stabilizing a pin passing through large through-holes 313. Through-holes 314 may be positioned on opposite faces of the pin post 310 with central axes that are orthogonal to central axes of the larger through-holes 313. In some examples, pin post 310 has a top surface 319 that is configured to be in contact with a surface of a ring 20, and a bottom surface 318 that is also configured to be in contact with a surface of a ring 20, such that either the top or bottom surface may be positioned against a ring surface. One or both of the top surface 319 and bottom surface 318 may include an opening 316 similar to opening 216 which is configured to receive a bolt or similar fastener to clamp the pin post 310 to the ring 20. As shown in FIGS. 5A-D, the top surface 319 and/or bottom surface 318 may include teeth 315. In this example, a total of two teeth 315 (per desired surface) may be provided and the teeth 315 may be substantially identical to teeth 215a in number and configuration. However, it should be understood that teeth 315 may be provided in different numbers and configurations, including numbers and configurations similar or identical to teeth 215b and/or 215c. It should be understood that any accessory configured to be in contact with a surface of a ring 20, 30 of an external fixation frame, where it is desirable for that accessory to resist rotation, may be provided with the friction-enhancing elements (e.g. teeth) described herein. Still other accessories may be modified to include the teeth described herein (or variations thereof), including for example on any sort of pin or wire adapters, or even on the ends of the telescopic struts themselves.

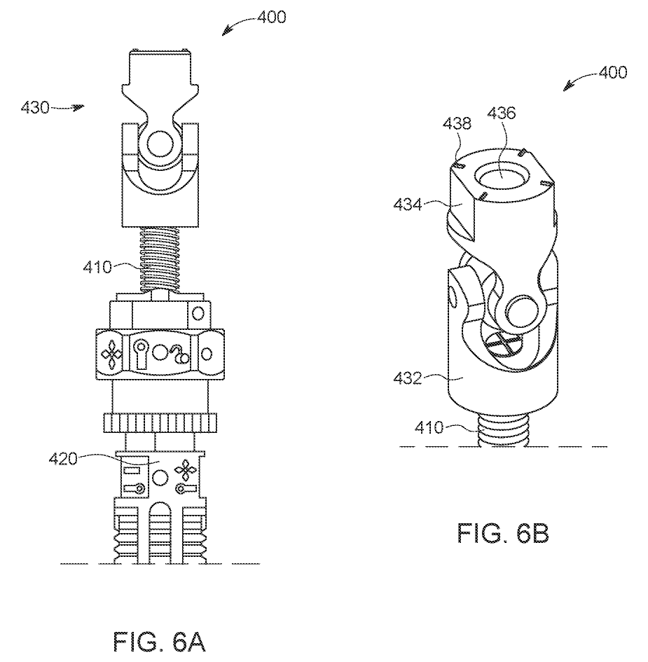

For example, FIG. 6A illustrate a strut 400 that may be similar or identical to struts 100a-f. Only one end of the strut 400 is shown in FIG. 6A, which may include a threaded rod 410 that extends into or out of tube 420 to increase or decrease the effective length of the telescopic strut 400. A first joint 430, which may be a universal joint, may be coupled to the threaded rod 410. The first joint 430 is shown in more detail in FIG. 6B. First joint 430 may include a bottom yoke 432 and a top yoke 434 (which may be connected via a rotating member not separately labeled in FIG. 6B). The top yoke 434 may include a bore 436 which may be configured to be positioned adjacent an opening in a ring (e.g. ring 20 or 30) and receive a threaded connected to fix the top yoke 434 to the ring. As shown in FIG. 6B, the top yoke may include friction enhancing elements, which may be in the form of protrusions or teeth 438. The teeth 438 may be configured to frictionally engage the ring in use, and may be substantially similar or identical to any of the other friction enhancing elements (or teeth or protrusions) described herein. It should be understood that another similar joint may be provided on the opposite end of the strut 400, such as at the end of the tube 420, for connection to the other ring (e.g. ring 20, 30), and similar friction-enhancing elements may be provided on that joint as well.

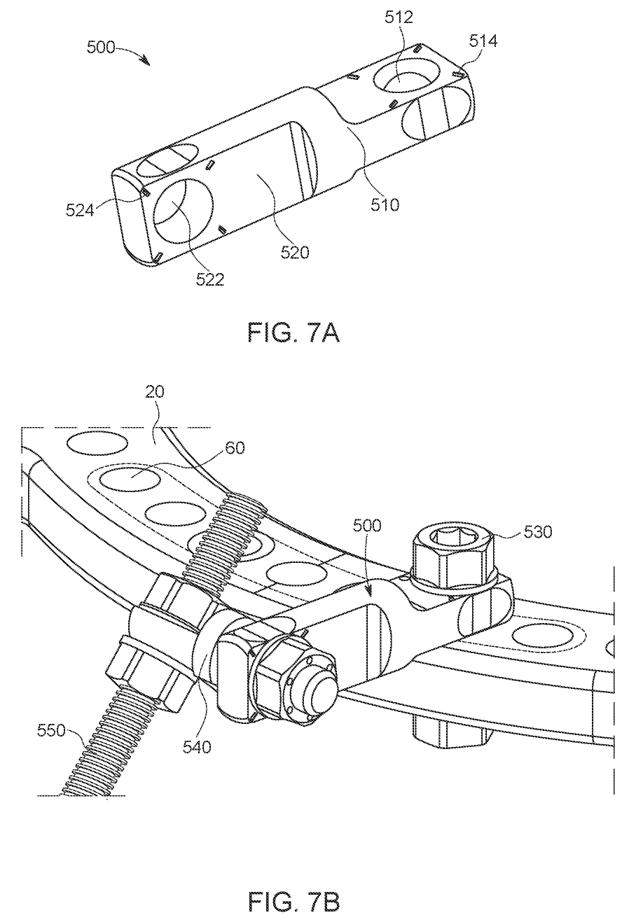

Another example accessory that may include friction enhancing elements is a twisted plate 500, for example as shown in FIG. 7A. Plate 500 may include a first flat extension 510 and a second flat extension 520, the two flat extensions each having a pair of flat sides, the pair of flat sides of the first extension 510 extending in generally orthogonal planes compared to the pair of flat sides of the second extension 520. Each flat extension 510, 520 may include a corresponding bore 512, 522 configured to receive a fastener therethrough to connect to another component, such as a ring (e.g. ring 20, 30) or to a further accessory component. As shown in FIG. 7A, one or both flat extensions 510, 520 may include a plurality of friction enhancing elements (e.g. protrusions or teeth 514, 524) around the bores 512, 522. The friction enhancing elements 514, 524 may be similar or identical to any of the other friction enhancing elements (e.g. protrusions or teeth) described herein, and may be used for substantially the same purposes. For example, FIG. 7B shows the plate 500 with the first flat extension 510 in contact with ring 20 and a fastener 530 extending through bore 512 and a corresponding aperture 60 in the ring 20 to fix the plate 500 to the ring 20. In this configuration, the teeth 514 are in contact with a portion of the ring 20 adjacent the corresponding aperture 60. The friction enhancing elements 514, 524 may be positioned on only one or on both sides of the flat extensions 510, 520, respectively. FIG. 7B shows one example of a connector 540 coupled to the second flat extension 520 via the bore 522, with a threaded rod 550 coupled to the connector 540, although other accessories may be used with plate 500 other than post 540 and rod 550.

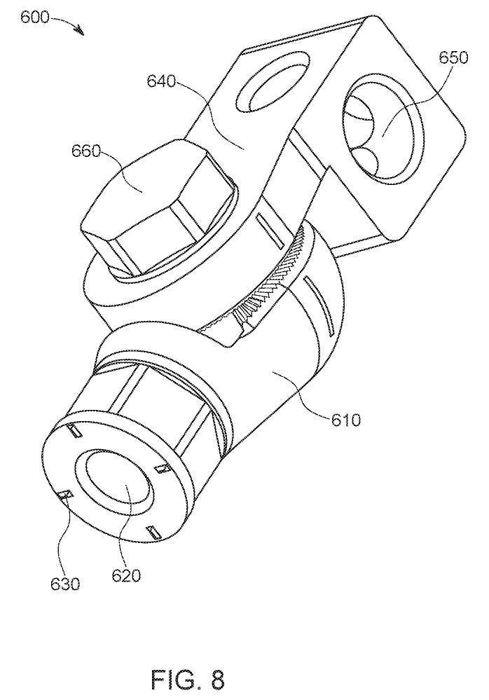

FIG. 8 is a perspective view of an example of an angled pin connector 600. The angled pin connector 600 may include a fixed portion 610 which includes a bore 620 configured to be positioned adjacent an opening in a ring (e.g. opening 60 in ring 20) and to receive a fastener (e.g. a threaded fastener) to secure the pin connector 600 to the ring. A plurality of friction enhancing elements (e.g. protrusions or teeth 630) may be positioned on the base of the fixed portion 610 around the bore 620 to help frictionally engage the pin connector 600 to the ring, in substantially similar or identical fashion as described for other friction enhancing members herein. The angled pin connector 600 may also include a rotating portion 640 that is rotatable relative to the fixed portion 610. The rotating portion 640 may include one or more apertures 650 for receiving a pin or other accessory therethrough. A fastener 660, which may be threaded, may connected the fixed portion 610 to the rotating portion 640. When the fastener 660 is loose, the rotating portion 640 may rotate relative to the fixed portion 610 to allow for the pin (or other accessory) secured to the rotating portion 640 have different positions and orientations. When the fastener 660 is tight, the rotating portion 640 may have a locked angular position relative to the fixed portion 610. The fixed portion 610 and/or the rotating portion 640 may include serrations, with the serrations engaging the other component (or engaging the serrations of the other component) to help enhance the angular locking upon tightening of fastener 660.

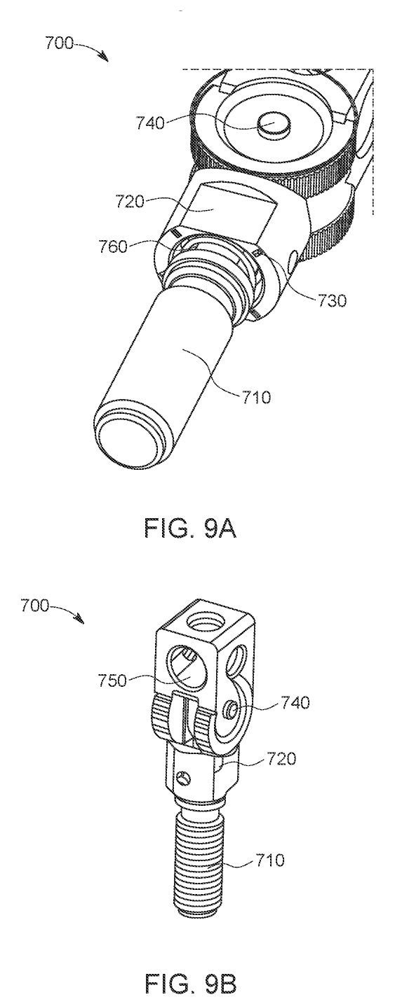

FIGS. 9A-9B are perspective views of an example of an angled pin adaptor 700 according to an example of the disclosure. Adaptor 700 may include a post 710 (which may be threaded) configured to extend through a hole of a ring (e.g. aperture 60 of ring 20), and a nut or other connector (not shown) may be used to secure the post 710 to the ring. As shown in FIG. 9A, the adaptor 700 may include a base 720 at an end of the post 710 (opposite the free or terminal end of the post 710) which may include an undersurface having a plurality of friction enhancing elements (e.g. protrusions or teeth 730), which may be similar or identical to the other friction enhancing elements described herein. In use, when the post 710 extends through the hole of the ring and a nut or other connector is threaded over the post 710 to secure the adaptor 700 to the ring 20, the undersurface of the base 720 contacts the ring, with the teeth 730 engaging the surface of the ring. The adaptor 700 may also include a rotating member 740 operably coupled to the base 720 and/or the post 710, with the rotating member 740 capable of rotating relative to the base 720 and/or the post 710. The rotating member 740 may include one or more openings 750 to receive another accessory component, such as a pin, so that the pin, when received in the openings 750, is capable of taking different rotational positions as the rotating member 740 rotates relative to the base 720 and/or the post 710. In some examples, the rotating member 740 includes serrations that contact an upper surface of the base 720, which may also have serrations. In some examples, a spring 760 or other biasing member may tend to push the upper surface of the base 720 into engagement with the rotating member 740 (e.g. the serrations of the upper surface of the baes 720 may be pushed against serrations of the rotating member 740 in the absence of applied forces). The spring 760 may tend to prevent rotation of the rotating member 740 by creating high frictional forces between the rotating member 740 and the base 720. If the user desires to rotate the rotating member 740 to adjust the angulation of the pin or other accessory received within the opening(s) 750, the user may pull the base 720 toward the post 710 to compress the spring 760 and to disengage the surface contact between the base 720 and the rotating member 740 to reduce friction that would otherwise prevent such rotation. Once the user releases force on the base 720, the spring 760 will tend to decompress to push the upper surface of the base 720 back into engagement with the rotating member 740 to lock against further relative rotation between the rotating member 740 and the base 720.

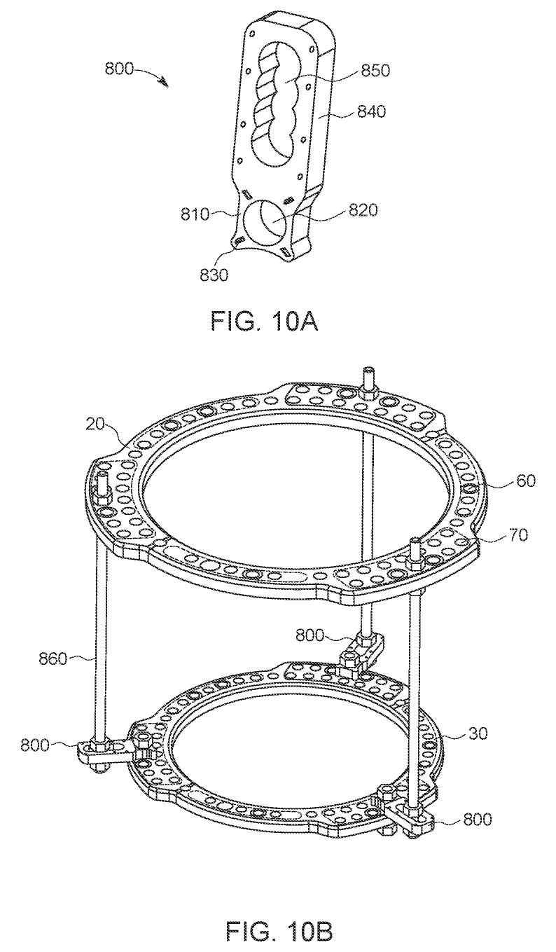

FIG. 10A is a perspective view of an example of a connection plate 800. The connection plate 800 may include a first end 810 which may include an opening 820 configured for placement adjacent an opening in a ring (e.g. opening 60 or 70 in ring 20 or 30). A plurality of friction enhancing elements (e.g. protrusions or teeth 830) may be positioned on the first end 810 around the opening 820. The teeth 830 may be substantially similar or identical to other teeth described herein, for the same or substantially the same purpose as described elsewhere. The teeth 830 may be positioned on one or both surfaces of the first end 810. The plate 800 may include a second end 840 opposite the first end 810, which may include one or more holes or openings 850 therein. In some examples, the openings 850 may be a single opening that are formed as portions of a circle or cylinder positioned in sequence, with each opening (or partial opening) configured to receive a rod or other accessory therethrough. For example, FIG. 10B shows two rings 20, 30 connected by rods 860. The rods 860 extend through openings 70 in the top ring 20 (and may be secured by nuts or similar fasteners) The bottom ring 30 may be smaller in diameter than the top ring 20, and thus the rods 860 may not be capable of extending directly through openings 60 or 70 in the bottom ring 30. Thus, a plurality of connection plates 800 are coupled to the bottom ring 30, with the first end 810 overlying the ring 30 and fasteners (not separately labeled) passing through both the opening 820 in the first end 810 and the opening 70 in the ring 30 to secure the connection plates 800 to the ring. As with other examples, the teeth 830 are configured to dig into the surface of the ring 30 which the first end 810 contacts for enhanced securement. The second ends 840 of the connection plates 800 may extend radially outward form the center of the ring 30, with the rods 860 passing through respective holes 850 in the second ends 840 of the connection plates 800. The rods 860 may be secured by nuts or other members to the second ends 840 of the connection plates 800 to produce a stable construct.

Although the invention herein has been described with reference to particular embodiments, it is to be understood that these embodiments are merely illustrative of the principles and applications of the present invention. For example, features described in relation to one particular embodiment may be combined with features of other embodiments described herein. It is therefore to be understood that numerous modifications may be made to the illustrative embodiments and that other arrangements may be devised without departing from the spirit and scope of the present invention as defined by the appended claims.

1. An external fixation system comprising:
a first fixation ring having a first plurality of holes extending therethrough;
a second fixation ring having a second plurality of holes extending therethrough;
a plurality of struts configured to couple the first fixation ring to the second fixation ring;
a post having a base, a free end opposite the base, first and second side walls each extending from the base to the free end, at least one hole extending from the first wall to the second wall through the post, a threaded hole having an opening positioned in the base and extending toward the free end of the post, and a first protrusion protruding from the base in a direction opposite the free end of the post; and
a threaded bolt,
wherein in an assembled condition of the external fixation system, the plurality of struts couple the first fixation ring to the second fixation ring, the base of the post is in contact with a top surface of the first fixation ring, and the threaded bolt extends through one of the first plurality of holes from a bottom surface of the first fixation ring into the threaded hole of the post to secure the post to the first fixation ring such that the first protrusion penetrates the top surface of the first fixation ring.

2. The external fixation system of claim 1, further comprising a second protrusion protruding from the base in the direction opposite the free end of the post, the first and second protrusions being positioned on opposite side of the opening of the threaded hole, wherein in the assembled condition of the external fixation system, the second protrusion penetrates the top surface of the first fixation ring.

3. The external fixation system of claim 2, wherein the first protrusion and the second protrusion each extend along a first imaginary line that also passes through a center of the opening positioned in the base.

4. The external fixation system of claim 3, further comprising a third protrusion and a fourth protrusion protruding from the base in the direction opposite the free end of the post, the third and fourth protrusions being positioned on opposite sides of the opening of the threaded hole.

5. The external fixation system of claim 4, wherein the third protrusion and the fourth protrusion each extend along a second imaginary line that also passes through the center of the opening positioned in the base, the first imaginary line being perpendicular to the second imaginary line.

6. The external fixation system of claim 5, wherein the base includes a first flat edge and a second flat edge parallel to the first flat edge, a first curved edge connecting the first flat edge and the second flat edge, and a second curved edge connecting the first flat edge and the second flat edge.

7. The external fixation system of claim 6, wherein the first, second, third, and fourth protrusions each terminate in a first end that is positioned along the first or second curved edge at a location adjacent to the first or second flat edge.

8. The external fixation system of claim 7, wherein the first, second, third, and fourth protrusions each terminate in a second end that is a located a spaced distance from the opening positioned in the base.

9. The external fixation system of claim 1, wherein the first fixation ring and the second fixation ring are each formed of carbon fiber.

10. The external fixation system of claim 1, wherein the first fixation ring and the second fixation ring are each formed of aluminum.

11. The external fixation system of claim 1, wherein the post is a pin post, and the system includes at least one pin configured to be received in the pin post and extend into bone of a patient.

12. The external fixation system of claim 1, further comprising a connector, the connector being configured to be received within the at least one hole of the post.

13. The external fixation system of claim 12, further comprising at least one pin configured to be received in the connector and extend into bone of a patient.

14. The external fixation system of claim 12, further comprising at least one wire configured to be received in the connector and extend through bone of a patient.

15. The external fixation system of claim 2, wherein in the assembled condition of the external fixation system, the first and second protrusions penetrate the top surface of the first fixation ring such that a bottom surface of the base is substantially flush with the top surface of the first fixation ring.

16. The external fixation system of claim 15, wherein during assembly of the external fixation system, the threaded bolt is configured to be tightened between about 11 Nm and about 17 Nm into the threaded hole of the post without deforming the one of the first plurality of holes.

17. A method of assembling an external fixation system, the method comprising:
coupling a first fixation ring to a second fixation ring via a plurality of struts, the first fixation ring having a first plurality of holes extending therethrough and the second fixation ring having a second plurality of holes extending therethrough;
positioning a base of a post on a top surface of the first fixation ring so that an opening in the base of the post aligns with one of the first plurality of holes;
passing a threaded bolt through the one of the first plurality of holes from a bottom surface of the first fixation ring toward the top surface of the first fixation ring until the threaded bolt is at least partially received within the opening in the base of the post; and
rotating the threaded bolt to engage threads of the threaded bolt with complementary threads of a threaded hole of the post extending from the opening in the base of the post in a direction toward a free end of the wire post to secure the post to the first fixation ring,
wherein during the rotating, a first protrusion extending from the base in a direction opposite the free end contacts with the top surface of the first fixation ring, and the rotating is continued until the first protrusion penetrates the top surface of the first fixation ring.

18. The method of claim 17, wherein during the rotating, the first protrusion and a second protrusion extending from the base in the direction opposite the free end contacts with the top surface of the first fixation ring at locations on opposite sides of the one of the first plurality of holes, and the rotating is continued until both the first and second protrusions penetrate the top surface of the first fixation ring.

19. The method of claim 18, wherein the first protrusion and the second protrusion each extend along a first imaginary line that also passes through a center of the opening in the base.

20. The method of claim 19, wherein the post includes a third protrusion and a fourth protrusion protruding from the base in the direction opposite the free end of the post, the third and fourth protrusions being positioned on opposite sides of the opening in the base.
